(19) United States
(12) Patent Application Publication
(10) Pub. No.: US 2004/0053349 A1
(43) Pub. Date: Mar. 18, 2004

(54) Direct selection of protein complexes relevant to physiological or diagnostic context of a biological specimen

(76) Inventor: Nathan Citri, Jerusalem (IL)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/344,581

(22) PCT Filed: Aug. 15, 2001
(86) PCT No.: PCT/IL01/00760

(30) Foreign Application Priority Data
Aug. 16, 2000 (IL) 137905

(51) Int. Cl. G01N 33/554; G01N 33/569; C12Q 1/37

(52) U.S. Cl. 435/7.32; 435/23

(57) ABSTRACT

The invention provides a process for establishing and for direct comparison of liganded protein profiles of biological samples comprising selectively removing potential permeability barriers by disrupting intact membranes present in the samples treating the samples thereafter so as to eliminate unliganded proteins and analyzing the remaining liganded proteins so as to detect any differences between the samples.

The present invention relates to a process for selecting liganded proteins within a liquid biological sample.

More particularly, the present invention relates to a methodology of detecting proteins interacting, or designed to interact with other molecules within cells, tissues or other biological specimens and in particular to a methodology seeking to establish complexant protein profiles that will display variations relevant to the changing context of a physiological, experimental or diagnostic investigation.

Whereas the number of genes in an organism is constant, its protein content presents a much larger set of ever changing composition. We are now witnessing the beginnings of a great new area of research named proteomics and defined as the simultaneous study of many proteins in order to clarify the function of a single state of a cell. The sheer numbers of different and varying proteins present enormous technological problems, which are currently addressed. One such problem is how to select from an overwhelmingly crowded scene a manageable sample of proteins relevant in a defined context. The current methods employ for that purpose the equivalent of gene chips, namely “microarrays” of surface-bound ligands, which serve as specific “baits” for proteins of interest, which are then desorbed and analyzed.

While “baiting” provides an essential tool for isolating context related proteins it has important limitations, which are inherent to that approach:

1) Baiting requires interactions on solid surfaces. This is a departure from conditions prevailing in vivo and is likely to distort conformational effects involved in physiological interactions.

2) Baiting represents one-to-one protein complexes. However, many physiological interactions involve multiple components, which, moreover, often require a predetermined binding order.

3) Baiting will miss all proteins for which no bait is available. Discovery of unknown proteins with unexpected properties is among the most challenging tasks, which require a new approach as described below.

The object of the present invention is to eliminate the problems associated with the use of baits for removing the proteins of interest from their physiological context By reversing the “baiting” approach on its head the present invention provides a process for removal of proteins other than those relevant to the context in which the proteins of interest are retained for investigation. The present approach is based on the following considerations. It has been well established that the native conformation of a protein is flexible and that such flexibility, termed “conformational adaptability” [Citri, Conformational adaptability in enzymes. Advances in Enzymology, Vol. 37, pp. 397-648, 1973], allows the protein molecule to form specific complexes with other protein or non-protein ligands. The complex formation involves a conformational change in the protein. In its ligand-induced conformation the protein displays marked changes in susceptibility to thermal and other conformation-disrupting treatments, and to proteolytic degradation. As a rule, physiological ligands confer relative stability on the binding protein. In other words it is possible to set conditions such that protein molecules are denatured, degraded and eliminated unless rescued by one or more specific ligands present in, or added to, the sample tested.

The methodology based on the above considerations offers a simple and direct alternative to the very costly and elaborate baiting microarrays systems and, by essentially adhering to the physiologic contexts avoids the pitfalls associated with immobilized ligand technology. Above all it provides unique tools for discovery by providing direct access to poorly defined and even totally unknown interactions in the living cell.

It will be obvious that direct access to the protein content of a cell enables direct comparison of the liganded protein profiles of two or more samples, said comparison being based on selective removal of unliganded proteins before the preexisting balance between liganded and unliganded proteins in the respective samples had been disturbed.

Thus, the present invention provides A process for direct selection of protein complexes of interest by proteolytic elimination of irrelevant proteins from biological samples containing components derived from cells, tissues and other biological sources said process comprising:

a) selectively removing potential permeability barriers by disrupting any intact membranes present in the samples;

b) selectively isolating liganded proteins by proteolytic degradation and elimination of unliganded proteins utilizing proteolytic enzymes; and

c) analyzing the remaining liganded proteins to identify the components thereof.

In preferred embodiments of the present invention said elimination of unliganded proteins is carried out with the aid of a protease of broad specificity. In especially preferred embodiments said protease is a product of Streptomyces griseus, sometimes referred to as Pronase.

In a preferred embodiment of the above procedure the elimination of the unliganded proteins is carried out by incubation with a protease with broad specificity such as Pronase, in which case the fragmentation of the unliganded protein is extensive enough to leave little or no trace of the degraded proteins in a plain electrophoretic run.

In a preferred embodiment of the present invention there is provided a process for selecting liganded proteins within a biological sample comprising:

a) removing reversibly binding protein ligands present in said liquid sample;

b) introducing selected ligands chosen to interact with their respective cognate proteins and to bind therewith to form stabilized liganded protein molecules, in said liquid sample;

c) treating the resulting liquid sample to eliminate unliganded proteins by proteolysis; and

d) selecting the remaining liganded proteins to isolate the protein component thereof.

In a further preferred embodiment of the present invention said biological sample further comprises ligands which are proteins and which form protein-protein complexes, said process further comprising the step of subjecting said protein-protein complexes to dissociation and separation before the carrying out of step b, thereby enabling said dissociated proteins to interact with selected ligands which are themselves proteins and which are introduced into said sample and chosen to interact with said dissociated proteins.

In a further preferred embodiment of the present invention said liganded proteins are not defined and the process serves to detect changes in complex protein profiles that are relevant to the physiological or pathological state of said biological sample, said process comprising detecting said liganded proteins in comparison with a control sample, e.g., establishing the absence or presence and the relative location of said liganded proteins in said sample, as compared to said control sample. Thus, e.g., the results are used to construct protein interaction profiles, which provide the basis for comparison with identically constructed profiles derived from samples in a different physiological context.

As explained hereinbefore preferably said liquid biological sample contains components derived from cells, tissues or other biological sources and said process is utilized to establish complexant protein profiles of components within said sample.

In especially preferred embodiments of the present invention said resulting liquid sample is subjected to proteolytic degradation under conditions wherein unliganded proteins are eliminated, or denatured and eliminated and liganded proteins are retained.

In further preferred embodiments of the present invention said selected ligands are potential chemotherapeutic agents and the process serves to screen such agents by detecting losses in the stability of target proteins to denaturation and proteolysis, said losses resulting directly from binding of said selected ligands or indirectly from destabilization of target proteins by the competitive displacement of stabilizing ligands originally present in the biological sample, or added to that sample in said screening procedure.

As shown above, the present method can contribute to the search and design of new drugs by offering a simple and rapid screening procedure based on the principle that the ligand [e.g., a potential drug] will alter the conformation of the target protein. It is important to note that effective drugs are likely to be structural analogs of the natural ligands and, as such, are likely to promote rather than prevent denaturation and proteolysis of the target protein.

In U.S. Pat. No. 5,585,277 there is described and claimed a method for rapid screening to identify a ligand that binds to a predetermined target protein wherein the method identifies possible therapeutic test ligands by placing them in the presence of target proteins and determining the ability of tests ligands to increase the ratio of folded target protein to unfolded target protein. More specifically, said patent describes a method wherein a ligand for a target protein is identified by combining a test ligand with a target protein under conditions chosen to cause the protein to exist in an appropriate ratio of its folded and unfolded states in the case of a protein which unfolds reversibly or to cause the protein to unfold at an appropriate rate in the cases of a protein which unfolds irreversibly.

Thus, critical features of the method of said patent are to treat test and control combinations to cause the target protein in the controlled combination to unfold a measurable extent, determining the extent to which the target protein occurs in the folded state, the unfolded state or both in the test combination and in the control combination, comparing the determination made above between the test and control combination wherein if the target protein is present in the folded state to a greater extent in the test combination than in the control combination the test ligand is a ligand that binds to the target protein and repeating the process with the plurality with the test ligands until a ligand that binds to the target protein is identified.

In contradistinction, the present invention is based upon a method wherein if the target protein is present in the folded state to a lesser extent it is identified as a potential drug.

In other words, if the target protein is present in the unfolded state, as opposed to the folded state specified in said U.S. patent, to a greater extent in the test combination than in the control combination, the test ligand is also a ligand that binds to the target protein, but in contrast to other ligands, which favor folding and thereby increase the stability of the target protein and are selected for in the U.S. patent, said ligands which favor the unfolded state which is more readily degraded are, according to the present invention, selected by repeating the process of comparing the test combination and the control combination with the plurality of test ligands until a ligand that destabilizes the target protein is isolated. To emphasize: Ligands selected as drug candidates in the present invention would be discarded by the U.S. patent in its search for drug candidates, whereas according to the present invention the U.S. patent procedure must be reversed to select ligands which destabilize directly or indirectly the target protein and thus provide the desired drug candidates.

It will be obvious that detection of protein complexes displaying reduced stability to proteolytic degradation can be optimized by the use of a screening device based on the present invention and incorporating in a preferred embodiment a double-beam spectrophotometer yielding and recording differential profiles of protein-ligand interactions in the absence and presence of one or more proteases acting singly or in a selected combination or sequence.

In WO 99140435 (D1) there is described and claimed a method for identifying a protein folding inhibitor comprising contacting a protein biosynthetic system under protein synthesis conditions with at least one test compound and determining whether said test compound increases the ratio of unfolded protein to folded protein, wherein an increase in said ratio is indicative that said test compound is a protein folding inhibitor.

As is known to persons skilled in the art inhibitors of folding can only work in a living cell and only at the stage of protein synthesis. In contradistinction the present invention has nothing to do with protein synthesis and it is intended to be performed on proteins that are analyzed in a biological sample outside of any living cell. Furthermore essential features and requirements of said process include that the ligand must have the property of recognizing and binding to unfolded or partially folded proteins (page 5, lines 6-10) that the ligand must inhibit the folding (page 5, lines 6-18) that the inhibitor contact a biosynthetic system under protein synthesis conditions (page 7, lines 17-19) and that the inhibitor is one which specifically inhibits de novo folding (page 16, lines 6-7). Thus said patent neither teaches nor suggests the process of the present invention as defined in any of the claims herein.

U.S. Pat. No. 5,679,582 (D2) is directed to a method for identifying a ligand that binds a predetermined target protein. According to the method the target protein is incubated in the presence of a test ligand to produce a test combination, and in the absence of a test ligand to produce a control combination. The test and control combinations are then treated to cause a detectable fraction of the target protein to exist in a partially or totally unfolded sate. The extent to which the target protein occurs in a folded state, an unfolded state, or both, in the test and control combination is then determined. When the target protein is present in the folded state to a greater or lesser extent in the test combination than in the control combination, the test ligand is a ligand that binds the target protein (column 1, line 63 to column 2, line 9). Binding of the test ligand to the target protein is detected through the use of proteolysis (column 6, lines 28-62 and examples 1, 2, 6 and 10). As will be realized said method serves to identify a ligand of a predetermined target protein wherein a target protein refers to a peptide, protein or protein complex for which identification of a ligand or binding partner is desired (column 3, lines 16-18).

In contradistinction the present invention enables the selection and identification of components of complexes of both unknown proteins and unknown ligands since neither the ligand nor the target protein needs to be predetermined. Thus said patent neither teaches nor suggests the process of the present invention as defined in any of the claims herein.

DE 432327 (03) discloses a method for detection of protein-DNA complexes which are present in malignant cells, which shown uncontrolled proliferation (page 2, lines 56-58, page 3, lines 41-58). The protein complexes are isolated by lysing the cells, treating the resulting supernatant with a protease and isolating the complexes using cesiumchloride gradient cntrifugation (page 3, lines 19-27); Examples 1 and 2). Thus said patent deals exclusively with protein-DNA complexes with the aim of identifying potential tumor cells and is therefore totally unrelated to the present invention and does not teach nor suggest the presently claimed process for direct selection of protein complexes of interest by proteolytic elimination of irrelevant proteins from biological samples containing components derived from cells, tissues and other biological sources as defined in the specification claims.

WO 99/38013 (D4) refers to methods for comparing protein or protein-complex expression profiles and characterizing ligands of proteins present in biological samples. As will be noted this publication in fact relates to array-based technology as can be seen from the description on pages 5-8 and is the exact sort of technology which has been found to be unsatisfactory and which is intended to be replaced by the process of the present invention.

As will be realized the process of the present invention differs from the teachings of D4 in that a protease is used in the present invention to remove unliganded proteins which is neither taught nor suggested by said reference. Furthermore the underlying technical problem of the present invention is not providing an alternative method for assessing proteins bound to their ligands and instead is directed to a new approach for direct selection of protein complexes of interest even when the proteins are unknown said process being based on proteolytic, elimination of irrelevant proteins from biological samples containing components derived from cells, tissues and other biological sources as defined herein. Therefore none of the above mentioned references teach or suggest the subject matter of the present invention.

The present invention also provides a diagnostic kit for monitoring drugs targeting enzymes or other functional proteins in biological specimens, said kit comprising a selected target protein preparation, delivered in solution or in a soluble form, a protease preparation in solution or insolubilized and an indicator strip or solution reporting on catalytic, or other functional activity of said target protein following incubation of said target with said protease in said specimen as compared with identically performed test without protease being added.

It should be noted that a loss of native stability in general, and labilization to proteolysis in particular may occur without any evidence of binding. In other words, a protein may display an anomaly, most likely traceable to a mutation that impairs the correct folding of that protein. The present invention offers a direct approach to investigation and diagnosis of disorders presumed or known to be linked to protein anomaly. The application of the present method will be based on comparing the protein profiles of a healthy and diseased specimen after having both specimens subjected to appropriately calibrated proteolysis. The calibration is intended to ensure that correctly folded proteins are not degraded. If an anomalous protein is located, the natural ligand or ligands of said protein can be traced as in previously described applications, namely by their ability, however incomplete, to increase the stability of said anomalous protein to proteolytic degradation.

The applications of the new methodology and the preferred embodiments of the invention will be clarified with the aid of the following illustrative examples.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLE 1

### Contextual Selection of Enzymes in a Sample of Human Serum

Demonstration of Procedure and Recovery

1. A dialyzed serum sample is spiked with the 10 enzyme preparations listed in Table 1 hereinafter.

2. Ligand solutions (LS) are composed as follows:

One) ATP, XMP, CMP, NADP and NADPH are included in ligand solution LSA; and

Two) Glucose, L-aspartic acid, D,L-asparagine, creatine, D,L-glyceraidehyde-3-P are included in ligand solution LSB.

2. The serum is treated with trypsin in the absence and presence of LSA or LSB, or both. At the end of the treatment samples are assayed for enzyme activity. For details see Table 1.

TABLE 1

Relative residual activity
[percent of untreated]

| Enzyme | LSA* | LSB* | LSA + LSB* |
|---|---|---|---|
| Aldolase | 8 | 91 | 91 |
| Asparaginase | 9 | 89 | 90 |
| ATPase | 94 | 6 | 93 |
| Creatine kinase | 11 | 95 | 95 |
| Glucan | 9 | 94 | 93 |
| phosphorylase | | | |
| GMP synthetase | 89 | 11 | 90 |
| Glutamate DH | 98 | 12 | 98 |
| Homoserine DH | 95 | 9 | 95 |
| Isoleucyl t-RNA | 89 | 8 | 90 |
| synthetase | | | |
| Ribonuclease A | 93 | 11 | 93 |

*LSA or LSB or both are added before treatment with trypsin. For details of treatment see Zyk et al. [1969] Biochemistiy Vol. 8, pp. 2787-2794. Residual enzyme activities are assayed as in respective references cited by Citri [1973] Adv. Enzymol. Vol. 37, pp. 473-477 [Table ix].

Comments

The above Example is meant to illustrate the efficiency of the proposed selection procedure. In actual applications of this method the identity of the interacting molecules [“interactants”] contributed by either the protein [P or the ligand [LS] moiety, or both remains to be established as shown in the following Examples.

### EXAMPLE 2

### Contextual Selection of Serum Proteins

Procedure and Comments

In this example the samples of serum and the ligand solutions are as in Example 1. However, the serum profile is established on the basis of the molecular, rather than catalytic, properties of the ligand-selected proteins. The structural identification by accepted methods [not shown] may be, in this case confirmed by the corresponding enzyme assays [see Example 1].

1. Standard methods are used to selectively remove the bulk protein fractions [albumin and globulins] before testing.

2. The serum is treated with trypsin as in Example 1. At the end of the treatment protein fragments are removed by standard gel filtration and the protein fraction resolved by standard techniques [SDS-PAGE or the 2-D version followed by MS].

### EXAMPLE 3

### Fractional Analysis of Cytosol Proteins: Rescued Protein Profiles

Procedure

1. Cytosol preparation derived from E. coli by any well-known procedure is fractionated by gel filtration. Fractions of mol. wt. of 15 K and up are pooled and marked [P], Remaining fractions [r1, r2, r3 . . . ] are retained to be tested, each in turn, for their effect on the pattern of rescue of proteins in [P].

2. Samples of [P] are treated with trypsin in the absence and presence of each of the test fractions [r1, r2, r3 . . . ] At the end of the treatment the proteins rescued by the respective ligand fractions are analyzed on gel by any of the established procedures.

3. The results are used to construct protein interaction profiles, which provide the basis for comparison with identically constructed profiles derived from cytosol in a different physiological context.

Comments

The profiles can be further refined by repeating the above procedure at a higher ligand resolution, which can be readily obtained by any conventional chromatographic separation method. It is important to note that this immediately suggests a direct and, so far the only, way of selecting and studying unknown interactions where none of the interactants, neither the protein nor its ligands, need to be known or even need to have been known to exist.

### EXAMPLE 4

### Fractional Analysis of Cytosol Proteins: Protein-Protein Profiles

Procedure

1. Cytosol preparation and derivation of protein pool [P] are as in Example 3 above.

2. Samples of [P] are subjected to proteolytic treatment by trypsin or other compatible proteases under 2 sets of conditions, namely:

One) ensuring dissociation of any protein-protein complexes present in [P], and

Two) favoring reassociation of dissociated complexes.

There is a choice of methods for achieving and maintaining dissociation and a choice of proteolytic enzymes for replacing trypsin as appropriate.

3. The protein-protein interaction profiles obtained by this procedure are constructed and used as in Examples 2 and 3 above.

Whereas in the examples hereinbefore the enzymic degradation of unliganded proteins was carried out by incubation with trypsin, other proteolytic enzymes may be employed for that purpose either instead of trypsin, as illustrated in the next paragraph, or as an added step in the process of selective proteolysis.

The choice of proteolytic enzymes depends on the procedure to be employed for removal of the degraded protein residues. An endopeptidase of wide specificity, such as chymotrypsin or pepsin, will produce a larger number of smaller fragments than that obtained with a protease of narrow specificity [e.g., trypsin]. Exopeptidases, such as carboxypeptidase, are likely to be advantageous for breaking down proteins denatured by treatment [e.g. controlled heat shock] which spares their liganded counterparts.

Further flexibility in the choice of proteases to suit the preferred procedure is enabled by the availability of proteases covering the entire range of pH values, starting with pepsin [pH 1.0-2.0] up to pH 10 with trypsin. Similarly, there is a wide range of temperature tolerance and there is a rich choice of detergent compatible proteases, such as subtilisins. An added factor in facilitating designs of procedure is the wide availability of immobilized or insolubilized protease derivatives.

### EXAMPLE 5

### A Kit for Rapid Detection of Traces of a Protein Targeted Drug

Components for Detecting an A-Type Betalactam Antibiotic:

1. Whatman #3 discs [4 mm dia] impregnated respectively with Un. penicillinase [Sigma]; b. pronase [Sigma].

2. Cefinase strips [commercially available detectors of penicillinase activity]

3. Test tubes [5×40 mm].

Procedure

1. Place a penicillinase disc in a test tube marked A, pronase in tube B and both discs in C and add 1.0 ml of liquid sample to be tested to each tube.

2. Incubate [4 min at 37° C.] and dip a cefinase strip in each tube.

Results

The strip will turn red in A confirming penicillinase activity. There will be no change of color in B. No change in color in cefinase strip dipped in C will indicate presence of a ligand, which promotes proteolysis of penicillinase [In this case 0.01 mcg of an A-type betalactam antibiotic].

Comments

1. In the Example above the kit provides a sensitive tool for instant detection of residues of an important class of antibiotics in circulation [for bedside monitoring] or in urine [for compliance tests].

2. This Example serves as an illustration of the principle that drugs such as competitive inhibitors of function of a target protein can be shown to alter the conformation of the cognate proteins and that such changes can be detected with much greater sensitivity than changes in function.

### EXAMPLE 6

### Screening for Compounds Relevant to Drug Discovery: Direct Binding Method

Procedure

1. Ligands: Methicillin [2.0 mg] or Pronase [2.0 mcg] or both were added to respective 1.0 mL samples of a 10% solution of casein hydrolyzate prepared in twice distilled water.

2. Targets: Penicillinase [50 mcg] was dissolved in 1.0 mL of a buffered solution containing 50 mcg of liver extract.

3. Incubation [10 min. at 37° C.] started with adding 0.1 mL of “targets” to each sameple of “ligands”.

4. The samples were analyzed by standard SDS-PAGE procedure.

Results

The Penicillinase Band [27 kD] was preserved intact in all lanes but one: it was completely missing in the lane derived from the sample incubated with both methicillin and Pronase.

Comments

This example illustrates an application of the current methodology to the isolation from a randomly produced population of molecules of candidate drugs targeting a defined protein. In the present example the target is penicillinase that has been added to a human liver extract. The candidate drug preparation is in this case represented by casein hydrolyzate to which a drug, methicillin, has been added. Methicillin is a structural analog of the natural ligand [substrate] of penicillinase. The analog binds to the same site as the natural ligand, but the resulting conformation is functionally disabled and also susceptible to proteolytic degradation under conditions where the free penicillinase molecule is spared. Hence, this example provides a model of a class of interactions that permit rapid screening of potential drug candidates.

### EXAMPLE 7

### Screening for compounds Relevant to Drug Discovery: Competitive Binding Method

Procedure

1. The ligand solution and the target preparation were as in Example 6, above.

2. A compound structurally related to he natural substrate of penicillinase, compound CT, was added [3.0 mg/mL] to half of the samples of the ligand solution

3. All other details including the SDS-PAGE analysis were as in Example 6.

Results

The results were as presented in the previous example in all lanes where CT was not added. The addition of CT had no effect except in one case, namely in the presence of both methicillin and Pronase. Whereas in the absence of CT the penicillinase band had disappeared [as in the previous example] in the presence of CT that band was preserved like in the control lanes lacking either methicillin or Pronase.

Comments

The effect of binding on susceptibility to proteolysis may not always be as striking as in the previous example. However, candidate drugs missed in the direct procedure illustrated above can be detected as illustrated here. The target protein, penicillinase, was included in the liver extract and compound CT which by itself has no obvious effect on the stability of penicillinase under the above conditions was added to the ligand solution. The presence of CT revealed itself only by counteracting the previously established effect of methicillin. The labilization that methicillin was expected to induce has been prevented by CT which competitively displaced methicillin. This illustrates an application of the present invention to the screening of molecules, which successfully compete for the specific ligand-binding site of the target protein. Such molecules are of the greatest interest in the field of drug discovery.

### EXAMPLE 8

### Screening for Compounds Relevant to Drug Discovery and Able to Enter the Intact Cell

Procedure

1. A preparation of an intact cell suspension derived from a prostate cell line was divided to two. One part was exposed to PBS containing DHS, a compound of potential interest [treated sample]. The other part was exposed to PBS alone [control sample].

2. Both samples were washed 6 times with PBS and analyzed as described in previous examples.

3. The profiles of the treated and the control sample were compared by the standard methods.

Results

1. Comparison of the protein profiles revealed that a band present in the control sample was nearly undetectable in the treated sample.

2. The results point to the following conclusions:

a) The cells that were investigated contain a protein, which binds and responds to DHS.

b) Although the cells were intact, the protein was accessible to DHS.

c) A similar analysis will provide the information on the location of that protein with respect to the cell membrane.

Comments

This example illustrates further unique advantages of the present methodology, namely the ability to screen for drug candidates in the natural context of the intact cell. No steps of protein expression, purification or other laborious procedures are required. Above all, the results of the direct screening illustrated here are as close to the natural context of the living cell as we can aspire.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A process for direct selection of protein complexes of interest by proteolytic elimination of irrelevant proteins from biological samples containing components derived from cells, tissues and other biological sources said process comprising:
a) selectively removing potential permeability barriers by disrupting any intact membranes present in the samples;
b) selectively isolating liganded proteins by proteolytic degradation and elimination of unliganded proteins utilizing proteolytic enzymes; and
c) analyzing the remaining liganded proteins to identify the components thereof.

2. A process according to claim 1 wherein said elimination of unliganded proteins is carried out with the aid of proteases of broad specificity.

3. A process according to claim 2 wherein said proteases are a product of Streptomyces griseus.

4. A process according to claim 2 for selecting liganded proteins within a biological sample comprising:
a) removing reversibly binding protein ligands present in said liquid sample;
b) introducing selected ligands chosen to interact with their respective cognate proteins and to bind therewith to form stabilized liganded protein molecules, in said liquid sample;
c) treating the resulting liquid sample to eliminate unliganded proteins by proteolysis; and
d) selecting the remaining liganded proteins to isolate the protein component thereof.

5. A process according to claim 4 wherein said biological sample further comprises ligands, which are proteins and which form protein-protein complexes, said process further comprising the step of subjecting said protein-protein complexes to dissociation and separation before the carrying out of step b, thereby enabling said dissociated proteins to interact with selected ligands which are themselves proteins and which are introduced into said sample and chosen to interact with said dissociated proteins.

6. A process according to claim 4, wherein the ligands are defined and serve to select and isolate both known and unknown cognate proteins.

7. A process according to claim 4, wherein said selected ligands are contained in a biological sample, which sample is added to said liquid sample.

8. A process according to claim 4, wherein said liganded proteins are not defined and the process serves to detect changes in complex protein profiles that are relevant to the physiological or pathological state of said biological sample, said process comprising selecting said liganded proteins for comparison with a control sample and the remaining liganded proteins of step (c) are selected to also isolate the ligand components thereof.

9. A process according to claim 4, wherein said liquid biological sample contains components selected from the group consisting of cells, tissues and other biological specimens and said process is utilized to establish complexant protein, profiles of components within said sample.

10. A process according to claim 4, wherein said resulting sample is subjected to proteolytic degradation under conditions wherein unliganded proteins are eliminated and liganded proteins are retained.

11. A process for selecting liganded proteins, wherein the protein ligands are potential therapeutic agents and the process serves to select complexes displaying losses of stability of target proteins to proteolysis, said losses resulting from binding of said ligands causing competitive displacement of stabilizing ligands present in, or selectively added to, the biological samples containing said target proteins.

12. A process according to claim 11 wherein said target proteins are structurally impaired and consequently deficient in their resistance to proteolysis.

13. A process for selecting liganded proteins within a liquid biological sample comprising:
a) introducing selected proteins chosen to interact with their respective cognate ligands and to bind therewith to form stabilized liganded protein molecules, in said liquid sample;
b) treating the resulting liquid sample by proteolysis to eliminate unliganded proteins; and
c) analyzing the remaining liganded proteins to identify the ligand component thereof.

14. A process according to claim 13, wherein the proteins are defined and serve to detect the presence and levels of both known and unknown cognate ligands.
